(19) United States
(12) Patent Application Publication
(10) Pub. No.: US 2025/0195816 A1
(43) Pub. Date: Jun. 19, 2025

(54) WATER DIVIDER FOR PREVENT REFLUX OF CONDENSATE WATER IN A VENTILATOR, AND RESPIRATORY CIRCUIT AND VENTILATOR USING THE SAME

(71) Applicant: RESVENT MEDICAL TECHNOLOGY CO., LTD, Shenzhen (CN)

(72) Inventors: Kai Qu, Shenzhen (CN); Rongrong Zhu, Shenzhen (CN)

(73) Assignee: RESVENT MEDICAL TECHNOLOGY CO., LTD, Shenzhen (CN)

(21) Appl. No.: 18/817,600

(22) Filed: Aug. 28, 2024

(30) Foreign Application Priority Data
Dec. 14, 2023 (CN) 2023117258768

(51) Int. Cl. A61M 16/08

(52) U.S. Cl. CPC A61M 16/0808; A61M 16/0883

(57) ABSTRACT

A water divider provided includes a tube body, and a gas splitting assembly. The water divider provides with a branch water tube, and a user tube opening, a drainage outlets, and an internal opening arranged in sequence along the branch water tube. The branch water tube is located between two ends of the main airway and communicated to the main airway. Gas exhaled by a user forms an airflow that flows from one end of the main airway into the branch water tube through the user tube opening, and flows out of the branch water tube from the internal opening into the other end of the main airway. The drainage outlet is connected an external liquid collection device. The gas splitting assembly locates in the branch water tube, and enables the airflow rotating when the airflow flows into the gas splitting assembly.

This non-provisional patent application claims priority under 35 U. S. C. § 119 from Chinese Patent Application No. 202311725876.8 filed on Dec. 22, 2023, the entire content of which is incorporated herein by reference.

### TECHNICAL FIELD

This application relates to ventilator technologies, in particular to a water divider for preventing reflux of condensate water in a ventilator, a respiratory circuit, and a ventilator.

### BACKGROUND

The problem of internal condensation water in ventilators has always been a concern for frontline health care works in their use of ventilators, especially use the ventilators with single circuit. However, the existing ventilator with single circuit have not address the internal condensate water.

### SUMMARY

In view of this, a low-cost and highly reliable water divider, a respiratory circuit, and a ventilator. for preventing reflux of condensate water in a ventilator are provided.

In a first aspect, a water divider for preventing reflux of condensate water in a ventilator provided includes a tube body, and a gas splitting assembly. The water divider includes a tube body, provided with a branch water tube, a user tube opening, a drainage outlet, and an internal opening arranged in sequence along the branch water tube, the user tube opening, the drainage outlets, and the internal opening are communicated with the branch water tube, the branch water tube is located between two ends of the main airway and communicated to the main airway through the user tube opening and the internal opening; gas exhaled by a user forms an airflow that flows from one end of the main airway into the branch water tube through the user tube opening, and flows out of the branch water tube from the internal opening into the other end of the main airway. The drainage outlet is configured to connected an external liquid collection device. The gas splitting assembly is located in the branch water tube, the gas splitting assembly enabling the airflow rotating when the airflow flows into the gas splitting assembly.

Secondly, a respiratory circuit provided includes a main airway and a water divider mentioned above to prevent the reflux of condensate water from the ventilator.

Thirdly, a ventilator provided includes a main body, a main airway provided by the main body, and a water divider to prevent the reflux of condensate water from the ventilator.

The above-mentioned ventilator employs the water divider to fully harness the properties of the exhaled gas from the user. It separates and condenses the high water content airflow, effectively preventing condensation from infiltrating the interior and causing corrosion to the device. Moreover, the water divider boasts no moving parts, ensuring high reliability in use. Additionally, the water divider it incorporates the separate collection device, that gathers the condensate water during operation, and dispenses it post-use, facilitating easy of operation and maintenance. Furthermore, the water divider can be design as a detachable structure, allowing for convenient removal for inhalation purposes or high-temperature disinfection through cooking, thereby offering excellent maintainability.

### BRIEF DESCRIPTION OF THE DRAWINGS

In order to provide a clearer explanation of the embodiments or technical solutions in the present application or prior art, a brief introduction will be given below to the accompanying drawings required in the embodiments or prior art description. It is evident that the accompanying drawings in the following description are only some embodiments of the present application. For those skilled in the art, other accompanying drawings can be obtained based on the structures shown in these drawings without creative labor.

FIG. 1 is a schematic diagram of a ventilator with a branch water tube provided in the first embodiment of the present application in the inhalation state.

FIG. 2 is a schematic diagram of a ventilator with a branch water tube provided in the first embodiment of the present application in the exhalation state.

FIG. 3 is a schematic diagram of the structure of the branch water tube provided in the present embodiment.

FIG. 4 is a schematic sectional view of the branch water tube shown in FIG. 3.

FIG. 5 is a schematic diagram of a ventilator with a branch water tube provided in the second embodiment of the present application in the inhalation state.

FIG. 6 is a three-dimensional exploded view of the branch water tube shown in FIG. 3.

FIG. 7 is a sectional view of the front stage vortex tube of the branch water tube shown in FIG. 6.

FIG. 8 is a three-dimensional view of the rear separation tube of the branch water tube shown in FIG. 6.

FIG. 9 is a sectional view of the rear stage separation tube shown in FIG. 8.

FIG. 10 is a schematic diagram of another angle of the branch water tube shown in FIG. 3.

FIG. 11 is another perspective view of the branch water tube shown in FIG. 3.

The implementation, functional characteristics, and advantages of the purpose of this application will be further explained in conjunction with the embodiments, with reference to the accompanying drawings.

### DETAILED DESCRIPTION OF THE EMBODIMENTS

In order to make the purpose, technical solution, and advantages of this application clearer and clearer, the following will provide further detailed explanations of this application in conjunction with the accompanying drawings and embodiments. It should be understood that the specific embodiments described here are only intended to explain the present application and are not intended to limit the present application. Based on the embodiments in this application, all other embodiments obtained by ordinary technical personnel in this field without creative labor fall within the scope of protection of this application.

The terms “first”, “second”, “third”, “fourth”, etc. (if any) in the specification and claims of this application, as well as the accompanying drawings, are used to distinguish similar planning objects and do not need to be used to describe specific order or sequence. It should be understood that the data used in this way can be interchanged in appropriate cases, in other words, the described embodiments are implemented in order other than those illustrated or described herein. In addition, the terms “including” and “having”, as well as any variations thereof, may also include other content, such as processes, methods, systems, products, or equipment that include a series of steps or units, not necessarily limited to those clearly listed, but may include other steps or units that are not clearly listed or inherent to these processes, methods, products, or equipment.

It should be noted that the descriptions related to “first”, “second”, etc. in this application are only for descriptive purposes and cannot be understood as indicating or implying their relative importance or implying the quantity of technical features indicated. Therefore, the features limited to “first” and “second” may explicitly or implicitly include one or more of the said features. In addition, the technical solutions between various embodiments can be combined with each other, but must be based on what ordinary technical personnel in the art can achieve. When the combination of technical solutions conflicts or cannot be achieved, it should be considered that the combination of such technical solutions does not exist and is not within the scope of protection required by this application.

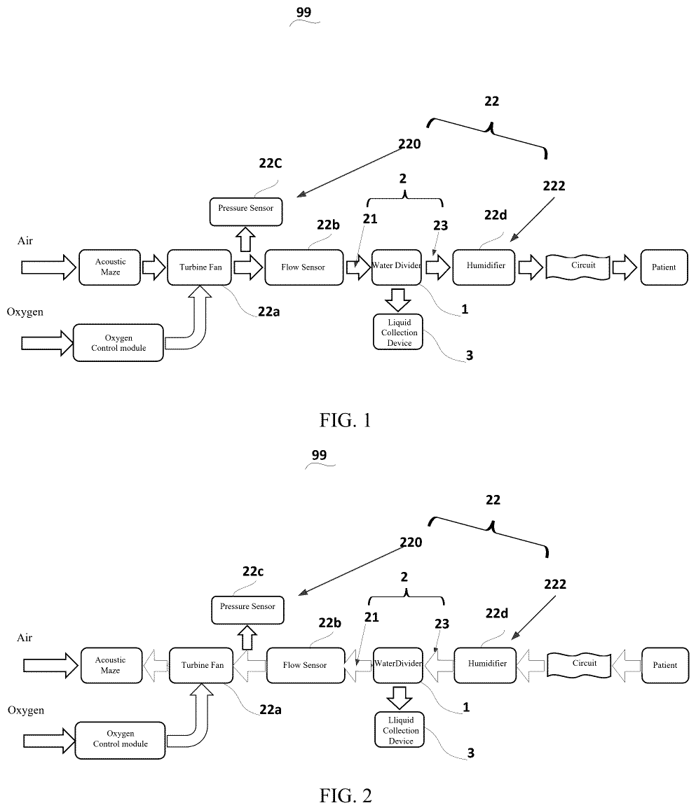

Referring to FIGS. 1-2, an ventilator 99 including a water divider 1 is illustrated. The ventilator 99 includes a main body 5 with a main airway 2. The ventilator 99 also includes a plurality of components 22 mounted in the main airway 2. The plurality of components 22 includes a turbine fan 22a, a flow sensor 22b, a pressure sensor 22c, a humidifier 22d and so on. The ventilator 99 includes an inhalation state and an exhalation status. The main airway 2 includes two ends, one end is an external interface for connecting to the outside world, and the other end is a user end. When the ventilator 99 is in the inhalation state, external gas enters into the main airway 2 from one end of the ventilator 99 (i.e. the external interface end) and flows along the main airway 2 to the other end (i.e. the user end) to assist the user in breathing, as shown in FIG. 1. When the ventilator 99 is in the exhalation state, the gas exhaled by the user enters into the main airway 2 from the user end and flows along the main airway 2, and is discharged from the external interface, as shown in FIG. 2. It can be understood that the temperature of the gas exhaled by the user is close to 37 degrees, which is higher than the temperature inside the ventilator 99. Therefore, the gas exhaled by the user will condense into liquid water when flowing through the interior of the ventilator 99. The users are those individuals who require assisted breathing, such as those who are unable to breathe autonomously or experience difficulty in breathing.

In this embodiment, the water divider 1 can be detachably connected between the two ends of the main airway 2 and communicated with the main airway 2, so that the gas exhaled by the user flows into water divider 1 from one end of the main airway 2, and the liquid water contained in the exhaled gas is discharged from the main airway 2 through water divider 1, and then flows from the other end of the main airway 2 to the plurality of components 22 in the main airway 2. It can be understood that the main airway 2 and the water divider 1 together form a respiratory circuit 21 of the ventilator 99. The water divider 1 connected to main airway 2 that main airway 2 includes a front main airway 21 and a rear main airway 23 separated by the water divider 1. Furthermore, it can be understood that the water divider 1 is detachably connected to the main airway 2 for easy cleaning, replacement, or maintenance. In some embodiments, the water divider 1 is fixedly connected to the main airway 2 if not considering replacement and maintenance.

In this embodiment, the plurality of the components 22 are divided into a first group of components 220 and a second group of components 222 based on whether the plurality of the components 22 are affected by liquid water or not. The first group of components 220 includes the components that are susceptible to the influence of liquid water, such as the flow sensor 22b and the pressure sensor 22c. The second group of components 222 includes the components that are not affected by liquid water, such as humidifiers. The first group of components 220 is arranged at the front main airway 20. It can be understood that when the ventilator 99 is in the exhalation state, the airflow of the front main airway 21 is the airflow obtained by the user's exhaled airflow being discharged from the liquid water by water divider 1, thereby avoiding damage to the flow sensor 22b and pressure sensor 22c by the liquid water.

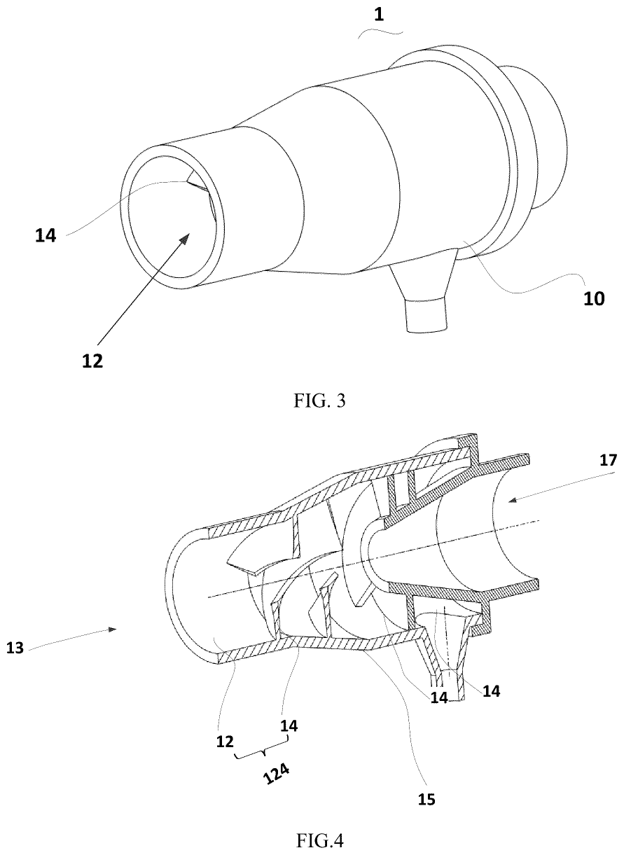

Referring to FIGS. 3-4, the water divider 1 includes a tube body 10 with a branch water tube 11 inside, swirling fins 12 located in the water divider 11, and separation fins 14. A user tube opening 13, a drainage outlet 15, and an internal opening 17 are defined in water divider 11 sequentially along water divider 11. That is to say, the drainage outlet 15 is set between the user tube opening 13 and the internal opening 17. The drainage outlet 15 is configured to connect to an external liquid collection device 3. The swirling fins 12 is located between the user tube opening 13 and the drainage outlet 15. The separation fins 14 is located between the swirling fins 12 and the internal opening 17. The gas exhaled by the user forms an airflow that flows sequentially from one end of the main airway 2 (i.e. the rear main airway 22) through the user tube opening 13, the water divider 11, and the internal opening 17, and then enters into the other end of the main airway 2 (i.e. the front main airway 21). In addition, the airflow entering water divider 1 is processed by the swirling fins 12 and the separation fins 14, and the liquid water contained in the airflow is separated and discharged from the drainage opening 15, thereby avoiding entering the front main airway 21 and avoiding damage to the devices (i.e. the first group of components 220) in the front main airway 21 due to the influence of liquid water.

In this embodiment, the liquid collection device 3 is a collection cup designed to gather the liquid water discharged from the discharge outlet 15. When the ventilator 99 is in operation, the water collection cup is mounted securely over the drainage opening 15, so that the water collection cup effectively sealing the drainage opening 15. So that the liquid collection device 3 maintains the drainage opening 15 in a sealed condition, ensures the integrity of the gas path and prevents air leakage.

In some other feasible embodiments, the water collection cup can be replaced by other water collection devices. For example, as shown in FIG. 5, the ventilator 99′ is provided in accordance with the second embodiment. The difference between the ventilator 99′ and the ventilator 99 is that the liquid collection device 3′ in the ventilator 99′ includes a drainage valve 31 and a drainage tube 32. The drain valve 31 is normally closed, the drain tube 32 and the drain valve 31 are design to control the connection and disconnection between drain tube 32 and drain outlet 15. In detail, when the ventilator 99 is in operation, the drain valve 31 is closed and maintain the drain opening 15 in a sealed condition; after the work is completed, the drainage valve is opened to drain the liquid water.

Refer to FIG. 5, in this embodiment, the tube body 10 includes a primary tube section 101, and a secondary tube section 102 assembled onto the primary tube section 101. The user tube opening 13, the drainage outlet 15, and the swirling fins 12 are located on the primary tube section 101, and together with the primary tube section 102 form a primary vortex tube 103. The internal opening 17 and the separation fins 14 are located at the secondary tube section 102, and together with the secondary tube section 102 form a secondary separation tube 105.

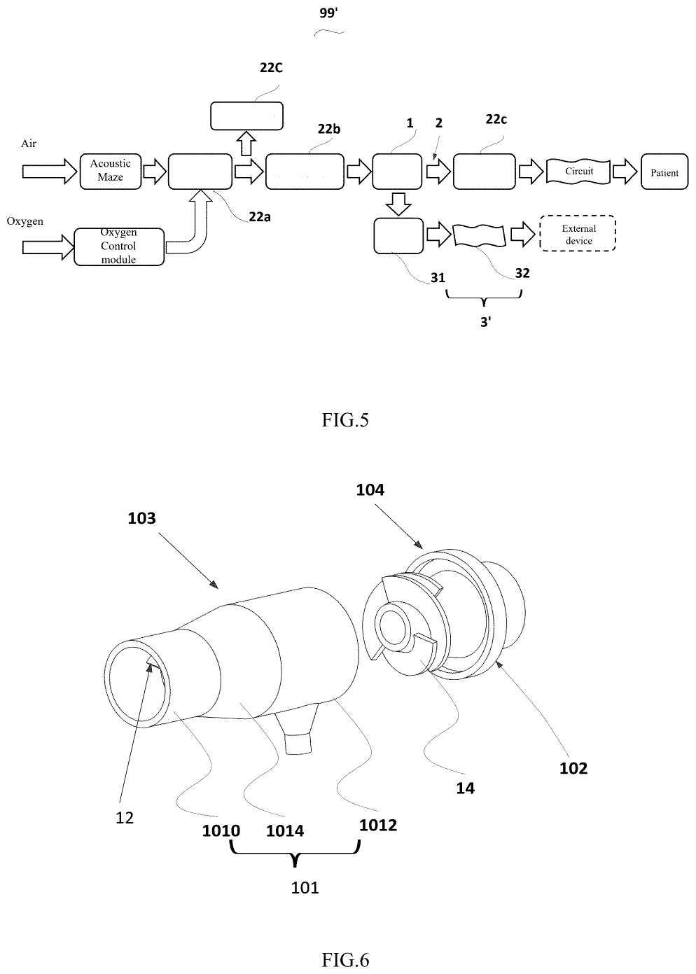

Referring to FIG. 6, the primary tube section 101 includes a primary small part 1010, and a primary large end 1012 arranged sequentially. The primary small part portion 1010 and primary large end 1012 are connected via a transition connection portion 1014. The user tube opening 13 is located at one end of primary small part 1010 away from primary large end 1012, and the central axis of the user tube opening 13 is collinear with a central axis of water divider 1, that is, the user tube opening 13 is coaxial with the water divider 1. The drainage outlet 15 is located at one end of the primary large end 1012 away from the primary small part 1010, and the central axis of drainage outlet 15 is perpendicular to the central axis of water divider 1. In this embodiment, the primary large end 1012 and the primary small part 1010 both being circular tubes, are coaxial and connected to each other, A diameter of the primary large end 1012 is greater than that of the primary small part 1010. The user tube opening 13 is defined on a free end of the primary small part 1010 that is not connected to the primary large end 1012, the drainage outlet 15 is defined on a wall of the primary large end 1012. The swirling fins 12 is located at a connection between the primary large end 1012 and the primary small part 1010, that is, located at the transition connection 1014.

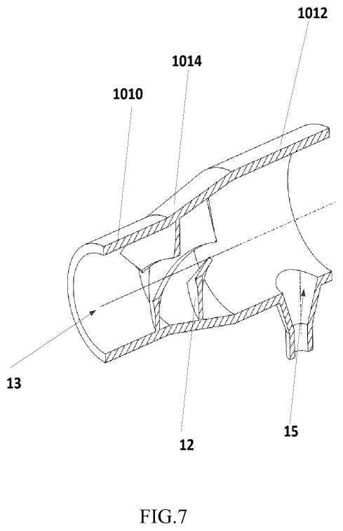

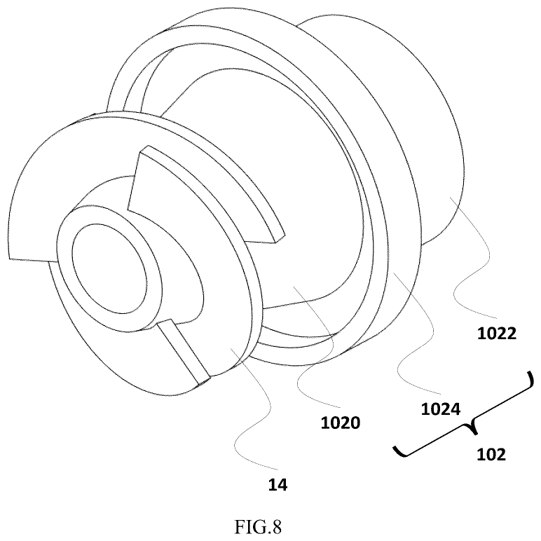

Referring to FIG. 7, the swirling fins 12 includes three fins that rotate inside the primary tube section 101 rotate under an action of the airflow to cause the airflow to rotate and stratify the rotated airflow. That is, the airflow with lower water content will be in a center position, and the airflow with higher water content will be near the wall of primary tube section 101. The airflow with higher water content comes into contact with the wall of the lower temperature primary tube section 101 and condenses into liquid water, which is then discharged from the drainage outlet 15 into the water divider 1. It can be understood that in some other embodiments, the quantity of the fins of the swirling fins 12 may vary with actual requirements, and can be two or four, without limitation.

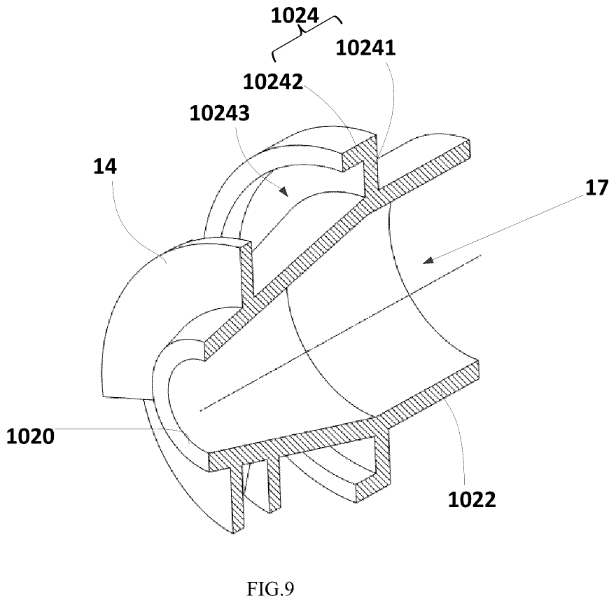

Referring to FIGS. 8 and 9, the secondary tube section 102 includes a secondary small part 1020 and a secondary large part 1022 arranged sequentially, and a sealing connector 1024 connected between the secondary small part 1020 and the a secondary large part 1022 for security connected to the primary large end 1012. the primary small part 1020 penetrates into the primary large end 1012. The secondary large part 1022 is located outside the primary tube section 101. The internal opening 17 is located at one end of the secondary large part 1022 away from the secondary small part 1020, the internal opening 17 and the user tube opening 13 are located at opposite ends of the water divider 1 and are coaxial with each other. The secondary small part 1020 is in the shape of a conical with openings at both ends. The small end 1021 of the secondary small part 1020 faces the user tube opening 13, and the large end 1023 of the secondary small part 1020 faces the internal opening 17. The secondary large part 1022 is cylindrical, and the sealing connector 1024 is located at the connection between the secondary small part 1020 and the secondary large part 1022.

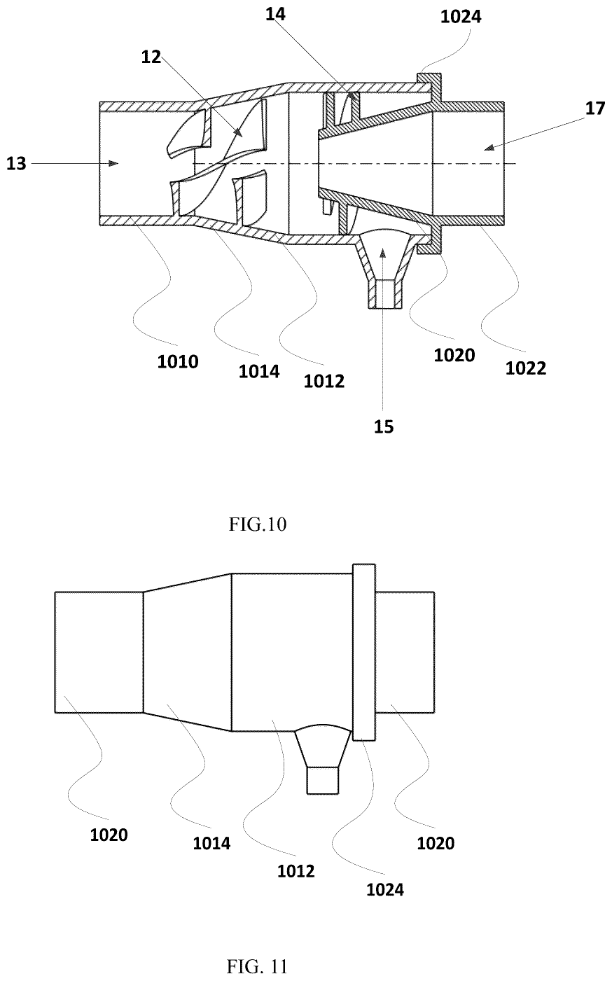

Referring to FIGS. 10 and 11, the separation fins 14 are located on an outer wall of the secondary small part 1020 near the small end 1021. The separation fins 14 include three spirally arranged fins. The airflow rotates under the guidance of the separation fins 14, and the rotation angle of the airflow at the separation fins 14 is greater than that of the vortex fins 12. Specifically, the airflow is guided by the swirling fins 12 to rotate and then flows into the separation fins 14. The separation fins 14 further accelerate the rotation and stratification, thereby throwing the airflow with higher water content further to the wall of the downstream tube 102 under an action of the separation fins 14. At this time, the airflow comes into contact with the wall of the secondary tube section 102 with lower temperature and condenses into the liquid water, which is discharged from the drainage outlet 15 and the water divider 1. Due to a certain gap between secondary small part 1020 and the primary large end 1012, the drainage outlet 15 is located on the wall of the primary large end 1012, thereby avoiding condensation water flowing along the main airway 2 and components 22 coming into contact. Furthermore, to prevent condensation water from flowing along the front main airway 21 and causing contact between the second group of components 222, thereby corroding the second group of components 222. It can be understood that in some other embodiments, the quantity of fins for separation fins 14 can vary with actual requirements, and can be two or four, without limitation.

It can be understood that the swirling fins 12 and the separation fins 14 rotate and stratify the airflow exhaled by the user, sequentially throwing the airflow with higher water content onto the wall of the water divider 1 to condense into the liquid water. In this embodiment, the swirling fins 12 and the separation fins 14 together form a splitting assembly 124 for rotating the airflow and then stratifying it. That is to say, the airflow with a higher water content, i.e. the airflow far from the center of the airflow, is thrown to the wall of water divider 1 to condense into the liquid water, and the liquid water is discharged from the outlet 15, so as to only allow the remaining airflow, i.e. the airflow located at the center of the airflow, to flow into the front main airway 21, avoiding the liquid water flowing towards the first group of components 220 in the main airway 2 and damaging the first group of components 220. It can be further understood that in some other embodiments, only the swirling fins 12 or the separation fins 14 may be included, which can also serve to condense liquid water from the wall of the water divider 1 when the airflow with high water content is thrown to the tube wall and discharged from the outlet 15.

The sealing connector 1024 includes a baffle 10241 that extends vertically around the wall of the secondary tube section 102 and a retaining wall 10242 that extends along the secondary tube section 102 towards the secondary small part 1020, surrounding an edge of the baffle 10241. The retaining wall 10242 is vertical and the small end 1021 is connected to the baffle 10241 inside, the baffle 10241, and the secondary tube section 102 to form an installation space 10243. Specifically, the baffle 10241 is a circular ring plate arranged around the secondary tube section 102. The retaining wall 10242 is a cylindrical wall with the same central axis as the secondary large part 1022. The installation space 10243 is configured to seal and connect one end of the primary large part 1012 away from the primary small part 1010, thereby limiting the airflow from the primary vortex tube 103 to only flow out from the secondary separation tube 105.

Referring to FIGS. 1-2 again, FIG. 1 is a schematic diagram of the airflow direction of ventilator 1 in the inhaled state. FIG. 2 is a schematic diagram of the airflow direction of ventilator 1 in the exhalation state.

When ventilator 1 is in the inhalation state, external air or oxygen flows sequentially from the external interface end in the first direction through turbine fan 22a, the flow sensor 22b, the water divider 1, and the humidifier 22d to enter into the user end, thereby assisting the user in inhaling. In detail, during the inhalation process, the external gas flows from the internal opening 17 of the water divider 1 to the user tube opening 13. That is to say, the gas flows directly from the inside of the secondary tube section 102 to the inside of the primary tube section 101 without acting on the separation fins 14, and the gas entering into the primary tube section 101 passes through the center of the primary tube section 101 and does not act on the swirling fins 12. Therefore, when the ventilator 1 in the inhalation state, the water divider 1 does not affect the function of assisting the user in inhalation.

When ventilator 1 is in the exhalation state, the gas exhaled by the user passes through the humidifier 22d, the branch water tube 1, the flow sensor 22b, and turbine fan 22a in the opposite direction to the first direction in sequence from the user end, and the exhaled gas is discharged from the external interface end.

The above-mentioned ventilator employs the water divider to fully harness the properties of the exhaled gas from the user. It separates and condenses the high water content airflow, effectively preventing condensation from infiltrating the interior and causing corrosion to the device. Moreover, the water divider boasts no moving parts, ensuring high reliability in use. Additionally, the water divider it incorporates the separate collection device, that gathers the condensate water during operation, and dispenses it post-use, facilitating easy of operation and maintenance. Furthermore, the water divider can be design as a detachable structure, allowing for convenient removal for inhalation purposes or high-temperature disinfection through cooking, thereby offering excellent maintainability.

Obviously, technical personnel in this field can make various modifications and variations to the present application without departing from the spirit and scope of the present application. In this way, if these modifications and variations of the present application fall within the scope of the claims and their equivalent technologies, the present application also intends to include these modifications and variations.

The above listed examples are only the preferred embodiments of this application, and of course, they cannot be used to limit the scope of the rights of this application. Therefore, the equivalent changes made according to the claims of this application still fall within the scope of this application.

1. A water divider for preventing reflux of condensate water in a ventilator, the ventilator comprising a main airway, the water divider comprising:
a tube body, provided with a branch water tube, a user tube opening, a drainage outlet, and an internal opening arranged in sequence along the branch water tube, the user tube opening, the drainage outlets, and the internal opening communicated with the branch water tube, the branch water tube located between the two ends of the main airway and communicated to the main airway through the user tube opening and the internal opening; gas exhaled by a user forming an airflow that flows from one end of the main airway into the branch water tube through the user tube opening, and flows out of the branch water tube from the internal opening into the other end of the main airway; the drainage outlet being configured to connected an external liquid collection device; and
a gas splitting assembly, located in the branch water tube, the gas splitting assembly enabling the airflow rotating when the airflow flows into the gas splitting assembly.

2. The water divider of claim 1, wherein the gas splitting assembly comprises swirling fins, which are located between the user tube opening and the drainage outlet. When the airflow flows towards the swirling fins, the swirling fins rotate to rotate the airflow.

3. The water divider of claim 2, wherein the gas splitting assembly further comprises a separation fin located in the branch water tube and between the swirling fins and the internal opening, the separation fins further rotate the airflow when the airflow passing through the rotating fin passes through the separation fins.

4. The water divider of claim 3, wherein the tube body comprises a primary tube section and a secondary tube section assembled on the primary tube section, the swirling fins, user tube opening, and the drainage outlet are arranged on the primary tube section to form a primary vortex tube, and the separation fins and the internal opening are arranged on the secondary tube section to form a secondary separation tube.

5. The water divider of claim 4, wherein the primary tube section comprises a primary large end and a primary small part arranged in sequence, the user tube opening is located at one end of primary small part far from primary large end, and primary large end is connected to the secondary tube section.

6. The water divider of claim 5, wherein the secondary tube section comprises a secondary large part and a secondary small part sequentially arranged, the secondary small part penetrates into primary large end, the secondary large part is located outside the primary tube section, and the internal opening is located at one end of the secondary large part far from the secondary small part.

7. The water divider of claim 6, wherein the separation fins are set on the an outer wall of the secondary small part, the secondary large part is located outside primary tube section, and the secondary small part is in a conical shape.

8. The water divider of claim 5, further comprising a sealing connector between the secondary large part and the small end of the rear stage for sealing and connecting the large end of the front stage.

9. A respiratory circuit for a ventilator, the respiratory circuit comprising:
a main airway, and
a water divider preventing reflux of condensate water in the ventilator, the water divider comprising:
a tube body, provided with a branch water tube, a user tube opening, a drainage outlet, and an internal opening arranged in sequence along the branch water tube, the user tube opening, the drainage outlets, and the internal opening communicated with the branch water tube, the branch water tube located between the two ends of the main airway and communicated to the main airway through the user tube opening and the internal opening; gas exhaled by a user forming an airflow that flows from one end of the main airway into the branch water tube through the user tube opening, and flows out of the branch water tube from the internal opening into the other end of the main airway; the drainage outlet being configured to connected an external liquid collection device; and
a gas splitting assembly, located in the branch water tube, the gas splitting assembly enabling the airflow rotating when the airflow flows into the gas splitting assembly.

10. The respiratory circuit of claim 9, wherein the gas splitting assembly comprises swirling fins, which are located between the user tube opening and the drainage outlet. When the airflow flows towards the swirling fins, the swirling fins rotate to rotate the airflow.

11. The respiratory circuit of claim 10, wherein the gas splitting assembly further comprises a separation fin located in the branch water tube and between the swirling fins and the internal opening, the separation fins further rotate the airflow when the airflow passing through the rotating fin passes through the separation fins.

12. The respiratory circuit of claim 11, wherein the tube body comprises a primary tube section and a secondary tube section assembled on the primary tube section, the swirling fins, user tube opening, and the drainage outlet are arranged on the primary tube section to form a primary vortex tube, and the separation fins and the internal opening are arranged on the secondary tube section to form a secondary separation tube.

13. The respiratory circuit of claim 12 wherein the primary tube section comprises a primary large end and a primary small part arranged in sequence, the user tube opening is located at one end of primary small part far from primary large end, and primary large end is connected to the secondary tube section.

14. The respiratory circuit of claim 13, wherein the secondary tube section comprises a secondary large part and a secondary small part sequentially arranged, wherein the secondary small part penetrates into primary large end, the secondary large part is located outside the front stage tube, and the internal opening is located at one end of the secondary large part far from the secondary small part.

15. The respiratory circuit of claim 14, wherein the separation fins are set on the outer wall of the small end of the rear stage, the large end of the rear stage is located outside the front stage tube, and the small end of the rear stage is in a conical shape.

16. The respiratory circuit of claim 13, wherein a sealing connector is provided at the connection between the large end of the rear stage and the small end of the rear stage for sealing and connecting the large end of the front stage.

17. A ventilator, comprises:
a main body;
a main airway located in the main body; and
a water divider for preventing reflux of condensate water in ventilator, the water divider comprising:
a tube body, provided with a branch water tube, a user tube opening, a drainage outlets, and an internal opening arranged in sequence along the branch water tube, the user tube opening, the drainage outlets, and the internal opening communicated with the branch water tube, the branch water tube located between the two ends of the main airway and communicated to the main airway through the user tube opening and the internal opening; gas exhaled by a user forming an airflow that flows from one end of the main airway into the branch water tube through the user tube opening, and flows out of the branch water tube from the internal opening into the other end of the main airway; the drainage outlet being configured to connected an external liquid collection device; and
a gas splitting assembly, located in the branch water tube, the gas splitting assembly enabling the airflow rotating when the airflow flows into the gas splitting assembly.

18. The ventilator of claim 17, wherein the gas splitting assembly comprises swirling fins, which are located between the user tube opening and the drainage outlet. When the airflow flows towards the swirling fins, the swirling fins rotate to rotate the airflow.

19. The ventilator of claim 18, wherein the gas splitting assembly further comprises a separation fin located in the branch water tube and between the swirling fins and the internal opening, the separation fins further rotate the airflow when the airflow passing through the rotating fin passes through the separation fins.

20. The ventilator of claim 19, wherein the tube body comprises a primary tube section and a secondary tube section assembled on the primary tube section, the swirling fins, user tube opening, and the drainage outlet are arranged on the primary tube section to form a primary vortex tube, and the separation fins and the internal opening are arranged on the secondary tube section to form a secondary separation tube.
